(19) United States
(12) Patent Application Publication
(10) Pub. No.: US 2021/0213050 A1
(43) Pub. Date: Jul. 15, 2021

(54) Compositions and Methods for the Removal of Extraneous Calcium Hydroxyapatite

(71) Applicant: HENRY FORD HEALTH SYSTEM, Detroit, MI (US)

(72) Inventor: David M. OZOG, Northville, MI (US)

(73) Assignee: HENRY FORD HEALTH SYSTEM, Detroit, MI (US)

(21) Appl. No.: 16/967,672

(22) PCT Filed: Feb. 5, 2019
(86) PCT No.: PCT/US2019/016573

§ 371 Date: Aug. 5, 2020

Related U.S. Application Data
(60) Provisional application No. 62/626,432, filed on Feb. 5, 2018.

(51) Int. Cl. A61K 33/04; A61K 9/00

(52) U.S. Cl. CPC A61K 33/04; A61K 9/0014; A61K 9/0019

(57) ABSTRACT

Described herein is a method for removing unwanted calcium hydroxylapatite or another calcium salt deposited by injection cutaneously in a subject, the method comprises administering an effective amount of a composition containing sodium thiosulfate to the deposited calcium hydroxylapatite or another calcium salt using a therapeutically effective regimen to diminish and or remove the amount of unwanted calcium hydroxylapatite or another calcium salt.

### CROSS-REFERENCE TO RELATED APPLICATION

This application claims priority under 35 U.S.C. 119(e) to provisional application Ser. No. 62/626,432, filed on Feb. 5, 2018, the entire content of which is incorporated by reference.

### TECHNICAL FIELD

The following examples of some embodiments of the invention are provided without limiting the invention to only those embodiments described herein and without disclaiming any embodiments or subject matter. The present disclosure relates to removal of undesirable calcium hydroxyapatite (or calcium hydroxylapatite) and other calcium salts embedded or deposited under the skin.

### BACKGROUND

In the cosmetic and dermatological setting, calcium hydroxylapatite or calcium hydroxyapatite (used interchangeably herein) injections (formulation of calcium hydroxylapatite (CaHA)) suspended in a sodium carboxymethylcellulose gel carrier have gained popularity as a means to fill in wrinkles and folds and may be used for tissue augmentation. CaHA is the primary ingredient for use as a wrinkle filler to plump the skin. CaHA is injected through a small needle and placed under the skin. Immediately, this filler works to add volume under the skin and over time, the benefits of CaHA continue by naturally stimulating your body's own natural collagen. In one example, dermatologically applied CaHA can include RADIESSE®. RADIESSE® is injected through a small needle and placed under the skin. Immediately, this filler works to add volume under the skin. RADIESSE® is known to have some side effects, including causing nodules, bumps or lumps in the back of the hand and other applied areas and can last up to a year.

However, with the increased use of CaHA and other filler materials like hyaluronic acid (HA) several patients have sought to have the injected CaHA or HA materials removed from their injection sites, perhaps due to improper implantation, skin or tissue allergy reactions and the like. The advantages of using HA as dermatological filler is that an enzyme called as hyaluronidase can be used to dissolve HA without further complications. The only way to remove unwanted CaHA is to perform surgery including forming an incision and excising, aspirating and otherwise physically removing the unwanted CaHA material. The only other option is to wait the 12-24 months for the product to be eliminated naturally, which may or may not be tolerable.

### SUMMARY

The following examples of some embodiments of the invention are provided without limiting the invention to only those embodiments described herein and without disclaiming any embodiments or subject matter. The present disclosure relates to removal of undesirable calcium hydroxylapatite and other calcium salts embedded or deposited under the skin.

In some embodiments, methods are provided for the removal of calcium bodies embedded within tissue of a patient in need thereof. In a first aspect the treatment includes administering a safe and therapeutically effective dose of a composition comprising sodium thiosulfate to the affected area containing the unwanted calcium body.

In some embodiments, a first aspect includes a method for removing unwanted calcium hydroxylapatite and other calcium salts, the method comprising administering a composition containing sodium thiosulfate to a subject to remove calcium hydroxylapatite or other calcium salt filler previously embedded under the skin which resulted in the formation of a lump, nodule, cyst, deformation, and/or an irregularity. In some embodiments, a composition comprising an effective amount of sodium thiosulfate can be applied topically or injected into the skin layers or tissue containing the unwanted calcium hydroxylapatite or other calcium salt filler. In some embodiments, multiple injections can be administered over a period of weeks to months to gradually remove the unwanted calcium hydroxylapatite, or other calcium salt filler.

### DETAILED DESCRIPTION

Without limitation, in some embodiments the present invention provides illustrative treatments comprising therapeutically effective compositions comprising sodium thiosulfate which when injected parenterally, for example through the skin into regions containing an unwanted calcification or unwanted calcium hydroxylapatite filler material.

To facilitate understanding of the disclosure set forth herein, a number of terms are defined below.

Generally, the nomenclature used herein and the laboratory procedures in inorganic chemistry, analytical chemistry, organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs. In the event that there is a plurality of definitions for a term used herein, those in this section prevail unless stated otherwise.

The terms “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject. In one embodiment, the subject has unwanted calcium hydroxylapatite deposition, and can be treated or ameliorated by the administration of sodium thiosulfate containing compositions as described herein.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The term “therapeutically effective amount” is meant to include the amount of a compound, such as sodium thiosulfate, that, when administered, is sufficient to treat the condition, or alleviate to some extent, one or more of the symptoms of the condition being treated. The term “therapeutically effective amount” also refers to the amount of a compound, such as sodium thiosulfate, that is sufficient to elicit the biological or medical response of a cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid (e.g., water, such as deionized or sterile water) or solid filler, diluent, excipient, solvent, or encapsulating material. In one embodiment, each component is “pharmaceutically acceptable” in the sense of being compatible with the other ingredients of a pharmaceutical formulation, and suitable for use in contact with cells, tissues, or organs of humans and animals without excessive toxicity, irritation, allergic response, immunogenicity, or other problems or complications, commensurate with a reasonable benefit/risk ratio. See, Remington: The Science and Practice of Pharmacy, 21st Edition, Lippincott Williams & Wilkins: Philadelphia, Pa., 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, Gibson Ed., CRC Press LLC: Boca Raton, Fla., 2004.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 5%, 4%, 3%, 2%, 1%, 0.5%, or 0.05% of a given value or range. In certain embodiments, it is contemplated that the values preceded by the term “about” or “approximately” are exact.

The terms “active pharmaceutical ingredient”, “active ingredient” and “active substance” refer to a compound, which is administered, alone or in combination with one or more pharmaceutically acceptable excipients, to a subject for treating, or ameliorating one or more symptoms of a condition, or disorder. As used herein, “active pharmaceutical ingredient”, “active ingredient” and “active substance” may be an optically active isomer of a compound described herein. As used herein, “active pharmaceutical ingredient”, “active ingredient”, and “active substance” may be the anhydrous, the monohydrate, dihydrate, trihydrate, quatrahydrate, pentahydrate, or other hydrated forms of sodium thiosulfate.

The term “sodium thiosulfate” includes anhydrous, monohydrate, dihydrate, trihydrate, quatrahydrate, pentahydrate, and other hydrated forms of sodium thiosulfate. In one embodiment, the “sodium thiosulfate” referred to herein is sodium thiosulfate (Na₂S₂O₃). In another embodiment, the sodium thiosulfate is pharmaceutical grade. The term “pharmaceutical grade” as used herein with respect to sodium thiosulfate means that the sodium thiosulfate was manufactured according to Good Manufacturing Practices (GMP) as detailed in the United States Code of Federal Regulations 21 CFR 211 and meets one or more of the purity levels recited herein.

The terms “drug,” “therapeutic agent,” and “active agent” refer to a compound, or a pharmaceutical composition thereof, which is administered to a subject for treating, preventing, or ameliorating one or more symptoms of a condition, disorder, or disease.

As used herein, an “effective amount” is defined as the amount required to confer a therapeutic effect or cosmetically effective effect on the treated patient, and is typically determined based on age, surface area, weight, amount of removal of CaHA needed, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, N.Y., 537 (1970). As used herein, “patient” refers to a mammal, including a human.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent. Among the acceptable vehicles, carriers, diluents and solvents that may be employed are water, Ringer's solution, U.S.P. and isotonic sterile saline or sterile sodium chloride solution.

Sodium Thiosulfate Compositions For Injection

In some embodiments, non-pharmaceutical, cosmetically acceptable compositions and/or pharmaceutically acceptable compositions are provided herein comprising sodium thiosulfate for administration to subjects, for example, human subjects with unwanted dermatological calcium hydroxyapatite (CaHA) deposition. In various embodiments, compositions comprising a concentration of sodium thiosulfate (molecular weight 158.11 g/mol) ranging from about 0.001M to about 0.5M dissolved in bacteriostatic saline or any other compatible physiological solution that may be administered via injection or other methods disclosed herein to layers of the skin and layers of the epidermis, dermis, and hypodermis. In some preferred embodiments, the concentration of the sodium thiosulfate administered to a subject in need thereof is about 12.5 mg/50 mL dissolved in a physiological and pharmaceutically acceptable excipient, carrier or diluent, for example, sterile saline (0.9% Sodium Chloride Injection, USP contains no preservatives).

In some embodiments, introduction of sodium thiosulfate compositions of the present disclosure into skin and tissue can include the use of liposomal carriers or other methods of encapsulating the sodium thiosulfate. In some embodiments, liposomes containing effective amounts of sodium thiosulphate can be administered into the various skin and cutaneous tissue layers to effectively supply sodium thiosulfate near unwanted CaHA deposits. In other embodiments, non-injectable administrations may be appropriate to introduce the sodium thiosulfate compositions to the location of the unwanted CaHA. In one illustrative embodiment, laser-assisted drug delivery (including fractional CO₂ laser/Er:YAG lasers) can be employed to introduce channels for delivery of concentrated sodium thiosulfate to areas containing unwanted CaHA for removal. Methods for performing laser-assisted drug delivery of sodium thiosulfate can be based on the teachings of Sklar, L. R. et al., (2014) “Laser Assisted Drug Delivery: A Review of An Evolving Technology”, Lasers Surg. Med. 46(4):249-62, the disclosure of which is incorporated herein by reference in its entirety. Other methods of abrading skin to facilitate penetration past epidermal barrier and iontophoresis methods for drug delivery may be used as is known in the art. The modern formulation technologies include, for example, microencapsulation and nanoencapsulation (matrix principle), membrane systems (e.g., liposomes) as well as cyclodextrins. Some of these methods are described, e.g., in the textbook Pharmazeutische Technologie: Moderne Arzneiformen, 2. ed., Wiss. Verl.-Ges. 1998 by Rainer H. Muller and Gesine H. Hildebrand, the entire disclosure of which is incorporated by reference herein in its entirety.

In some embodiments, cosmetic and pharmaceutically acceptable compositions can comprise sodium thiosulfate in concentrations ranging from about 0.001M to about 1M, or from about 0.01M to about 0.5M or from about 0.1 g/50 mL to about 500 g/50 mL, or from about 1 g/50 mL to about 100 g/50 mL, or about 10-20 g/50 mL or about 12.5 g/50 mL. These quantities of sodium thiosulfate can be dissolved in creams, solutions, suspensions, emollients, emulsions and the like. In some embodiments, sodium thiosulfate can be dissolved in a cosmetically acceptable dermatological cream and applied to the skin after using various rollers to introduce pinpoint holes through stratum corneum, epidermis and dermis, facilitating topical application of more concentrated sodium thiosulfate (for example, 0.01 to 1.0M concentrations of sodium thiosulfate). Suitable formulations for topical or cutaneous use, must be cosmetically or therapeutically effective and is not toxic to the host to whom it is administered. In particular, pharmaceutically acceptable carriers that are suitable for a sodium thiosulfate containing composition according to the present disclosure can include excipients, diluents and the like that are particularly suitable for the application of the composition over the skin, an orifice, or for injections into the epidermis, dermis, and hypodermis of a subject needing removal of unwanted CaHA, particularly facial deposition of CaHA as used in facial augmentation and the like.

For example, dermatological formulations for use in topical or injectable formulations it is known to those of skill in the art that such formulations are usually prepared with one or more auxiliary agents and additives. The cosmetic formulations according to the present disclosure can accordingly further contain cosmetic auxiliary agents as are conventionally used in such preparations; for example builders, preservatives, stabilizers, fillers, perfumes, pigments with/without coloring effect, thickeners, surface-active substances, emollients, moisturizers and/or humectants, anti-inflammatory substances, additional active agents, such as vitamins or proteins, light protection agents, insect repellents, bactericides, virucides, water, salts, antimicrobial, proteolytic or keratolytic substances, medicaments or other customary constituents of a cosmetic or dermatological formulation, such as alcohols, polyols, polymers, foam stabilizers, organic solvents and also electrolytes.

Cosmetic and/or dermatological formulations containing sodium thiosulfate according to the present disclosure can also be present as gels or creams which in addition to an effective amount of the sodium thiosulfate used in accordance with the present disclosure can also include solvents conventionally used for the preparation of same, usually and preferably one or more of sterile water, sterile saline and each of which may also contain organic and/or inorganic thickeners. Furthermore, these thickeners are a constituent of cosmetic emulsions.

As emulsifiers for producing the preparations according to the present disclosure, which may advantageously be applied to the desired areas of the skin as liquid or solid preparations, and which can be used in the preparations in small amounts, for example, from about 1% to about 6% by weight, nonionic types have proven to be suitable, such as polyoxyethylene fatty alcohol ethers, for example cetostearyl alcohol polyethylene glycol ether having 12 or 20 ethylene oxide units per molecule, cetostearyl alcohol and sorbitan esters and sorbitan ester-ethylene oxide compounds (for example, sorbitan monostearate and polyoxyethylene sorbitan monostearate), and long-chain higher molecular weight waxy polyglycol ethers. In addition, however, a large number of other emulsifiers or emulsifier mixtures are also suitable, which are normally used in cosmetic preparations. Non-limiting examples thereof include glyceryl stearate citrate, PEG 40 stearate or also polyglyceryl(3)-methylglucose distearate, stearic acid, steareth-2, steareth-21, glyceryl isostearate isoceteth-20 or also ceteareth-20.

In some embodiments, the oil (lipid) phase of the illustrative sodium thiosulfate containing formulations can be advantageously chosen from esters of saturated and/or unsaturated, branched and/or unbranched alkanecarboxylic acids having a chain length of from about 3 to about 30 carbon atoms and saturated and/or unsaturated, branched and/or unbranched alcohols having a chain length of from about 3 to about 30 carbon atoms, from esters of aromatic carboxylic acids and saturated and/or unsaturated, branched and/or unbranched alcohols having a chain length of from about 3 to about 30 carbon atoms. Such ester oils can then advantageously be chosen, e.g., from isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semi-synthetic and natural mixtures of such esters, e.g., jojoba oil.

Furthermore, the oil phase can be advantageously chosen from branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids having a chain length of from about 8 to about 24, in particular from about 12 to about 18 carbon atoms. The fatty acid triglycerides can, for example, be advantageously chosen from synthetic, semi-synthetic and natural oils, e.g., olive oil, sunflower oil, soya oil, peanut oil, rapeseed oil, almond oil, palm oil, coconut oil, palm kernel oil and the like.

Any blends of such oil and wax components can also be used advantageously for the purposes of the present invention. The oil phase can advantageously also contain cyclic or linear silicone oils or can consist entirely of such oils, although it is preferable to use an additional content of other oil phase components in addition to the silicone oil or silicone oils. Cyclomethicone (octamethylcyclotetrasiloxane) or dimethicone is advantageously used as the silicone oil for use according to the present invention. However, other silicone oils can also be advantageously used for the purposes of the present invention, for example hexamethylcyclotrisiloxane, polydimethylsiloxane or poly(methylphenylsiloxane).

In some illustrative embodiments, the concentration of the oil phase is from about 1% to about 50% by weight, based on the total weight of the preparations, preferably from about 2.5% to about 30% by weight, in particular preferably from about 5% to about 15% by weight.

Gelling agents, also called thickeners, are macromolecules which have a largely linear structure and have intermolecular forces of interaction which permit secondary and primary valence bonds between the individual molecules and thus the formation of a network-like structure. Some of them are water-soluble natural or synthetic polymers which form gels or viscous solutions in aqueous systems. They increase the viscosity of the water by either binding water molecules (hydration), or else by absorbing and encapsulating the water into their interwoven macromolecules, at the same time restricting the mobility of the water. Such water-soluble polymers represent a large group of chemically very different natural and synthetic polymers whose common feature is their solubility in water or aqueous media. A prerequisite for this is that these polymers have a number of hydrophilic groups sufficient for the solubility in water and are not too strongly crosslinked. The hydrophilic groups may be nonionic, anionic or cationic in nature.

Advantageous thickeners for cosmetic preparations are, for example, copolymers of C₁₀₋₃₀ alkyl acrylates and one or more monomers of acrylic acid, methacrylic acid or esters thereof. The INCI designation for such compounds is “acrylates/C₁₀₋₃₀ alkyl acrylate crosspolymer.” Particularly advantageous are the Pemulen®grades TR1, TR2 and TRZ by Goodrich (Noveon).

Carbopols are also advantageous gelling agents for the sodium thiosulfate containing preparations according to the present disclosure. Carbopols are polymers of acrylic acid, in particular also acrylate-alkyl acrylate copolymers. Advantageous polymers are, for example, the grades 980, 981, 984, 1342, 1382, 2984 and 5984, likewise the ETD grades 2001, 2020, 2050 and Carbopol Ultrez 10, PVM/MA decadiene crosspolymer (trade name Stabileze 06), polyglyceryl methacrylate, and polyacrylamide. Also advantageous gelling agents for such preparations are xanthan gum, polyvinylpyrrolidone, cellulose derivatives, in particular cellulose ethers, such as, for example, hydroxypropylmethylcellulose, starch and starch derivatives, hyaluronic acid, carob seed flour, silica and aluminum silicates.

The thickener usually is contained in a gel, a dispersion, an emulsion and the like in a concentration of from about 0.01% to about 5% by weight, preferably from about 0.1% to about 2% by weight, based on the total weight of the composition.

Suitable pharmaceutically acceptable carriers are well known in the art and are described for example in Remington's Pharmaceutical Sciences (Mack Publishing Company, Easton, USA, 1985), a standard reference text in this field. Pharmaceutically acceptable carriers are selected in accordance with the topical or injectable mode of administration, and the solubility and the stability of sodium thiosulphate.

Methods of Use

In some embodiments, cosmetic and pharmaceutically acceptable compositions containing sodium thiosulfate in concentrations ranging generally from about 0.001M to about 1.0 M, preferably from about 0.001M to about 0.5M are prepared using pharmaceutically acceptable practices. If an injectable formulation is prepared, the formulation may comprise 0.001M to about 0.5M sodium thiosulfate, and one or more cosmetic or pharmaceutically acceptable excipients, for example, sterile 0.9% sodium chloride solution. In various embodiments, the subject, preferably a human subject is in need or desires the removal of unwanted CaHA applied cutaneously to areas of the body such as the face or hands. The physician or cosmetologist may facilitate application of the sodium thiosulfate in injectable form after the identification of the unwanted CaHA is made. In some embodiments, radiological screening (for example, x-rays) may facilitate identification and localization of the unwanted CaHA. Next the physician or cosmetologist applies a sample of the sodium thiosulfate formulation to the unwanted CaHA. Typically an excess of the sodium thiosulfate formulation is applied proximate to the unwanted CaHA such that the majority of the surface area of the unwanted CaHA is in contact with the sodium thiosulfate formulation.

In some embodiments, a method for removing unwanted calcium salt, for example, CaHA, deposited by injection to the skin layers, for example, cutaneously in a subject. The method employs the general steps of administering an effective amount of a cosmetically acceptable composition containing sodium thiosulfate to the deposited calcium salt. In this method, the volume of the composition containing sodium thiosulfate is such that the entire volume of the unwanted calcium salt is in contact with the composition. In some embodiments, the unwanted calcium salt is calcium hydroxylapatite, for example, the calcium hydroxylapatite found in the FDA approved product Radiesse® which is manufactured and sold by Merz North America Inc, North Carolina USA. This product is approved by the FDA for the aesthetic use for the correction of moderate to severe facial wrinkles and folds and for the correcting of facial fat loss in people with HIV.

In some embodiments, the sodium thiosulfate composition contains an amount of sodium thiosulfate ranging from about 0.001M to about 1.0 M, or from about 0.001M to about 0.5M sodium thiosulfate, and one or more cosmetic or pharmaceutically acceptable excipients as described herein for topical, or injectable intradermal or subdermal administration, for example for injection into the subject's epidermis, dermis, or hypodermis layers to remove the unwanted calcium salt (e.g. CaHA).

In various embodiments, the method for removing unwanted calcium salt from a subject's skin can include administration of sodium thiosulfate to the unwanted calcium salt such that after administration of the sodium thiosulfate, a ratio of sodium thiosulfate to calcium salt (wt/wt) ranges from about 0.01 to 1 to about 1000 to 1. At these doses, the unwanted calcium salt (e.g. CaHA) is at least 50% surrounded by the sodium thiosulfate containing composition. In some embodiments, an exemplary volume of about 0.01 mL to about 1.0 mL of unwanted calcium salt is injected intralesionally with about 0.1 mL to about 3.0 mL of the sodium thiosulfate containing composition. In these illustrative uses, the amount of sodium thiosulfate in the sodium thiosulfate containing composition administered to remove the unwanted calcium salt (e.g. CaHA) ranges from about 1 g/50 mL to about 50 g/50 mL. In related embodiments, the unwanted calcium salt to be removed is calcium hydroxylapatite, for example, the CaHA found in RADIESSE®.

While the treatment to remove the unwanted calcium salt (e.g. CaHA) can be applied immediately after administration of the calcium salt, such as when a medical or cosmetic professional realizes that an excess of calcium salt (e.g. CaHA) has been applied, or applied in the incorrect location, for example when applied inadvertently via intravascular injection leading to possible vascular compromise, nodule formation or overcorrection, the method for removing such unwanted calcium salt (e.g. CaHA) can include administering the sodium sulfate containing composition immediately, after 1 to 30 minutes, after 1 hour, after 1 to 7 days, after 1-12 weeks, after 3-12 months or after 1 to 3 years, after the deposition of the calcium salt. The frequency and volume can be determined empirically, and can be influenced by the size of the volume of unwanted calcium salt to be removed, the location of the unwanted salt and other factors known to experienced medical and cosmetic professionals. In some exemplary methods the sodium thiosulfate containing composition is administered one to ten times topically, via injection or a combination of both at the site of the unwanted calcium salt. For example, the composition containing the sodium thiosulfate can be administered in the described volumes and concentrations to remove the unwanted calcium salt deposited subdermally, for example, the unwanted calcium salt is deposited in the epidermis, dermis, or hypodermis of the face, ears, or hands to correct various defects in the subject for which application of calcium salts (e.g. CaHA) is known. In some embodiments the correction requires removal of the unwanted calcium salt which was deposited in the face to correct wrinkles, folds, lips, or facial fat loss.

In some embodiments, methods for priming the surface of the skin to accept the sodium thiosulfate formulation can be used. In some embodiments, laser-assisted drug delivery (including fractional CO₂ laser/ErYAG laser) can be used to introduce channels for delivery of concentrated sodium thiosulfate containing formulations, suspensions, emulsions and compositions. In other embodiments, any or all methods of abrading skin to facilitate penetration past epidermal barrier, can be used prior to topical application of a sodium thiosulfate formulation. As used herein, topical application can encompass all administration and application forms for external application to human skin. Topical application comprises topical external use or application, such as the application as cream, lotion, gel, ointment, tincture, skin oil, milk, balm, by means of plaster, cloth, textile, pad, as spray, as an atomizer, aerosol, roll-on, stick, soft solid, powder, powder spray and other known forms of topical application, such as, e.g., insertion into the skin by an iontophoresis apparatus, ultrasound, microchannels or microneedles and the like. With the latter methods, a distinct reinforcement of penetration is achieved, which can lead to an intensified effect. Dermal rollers may also be used to introduce pinpoint holes through stratum corneum, epidermis and dermis, facilitating topical application of more concentrated sodium thiosulfate formulations as described herein.

### EXAMPLES

### Example 1. Removal of Calcium Hydroxylapatite in an Experimental Model

In an experimental model of removal of unwanted CaHA, an exogenously introduced product such as calcium hydroxylapatite in 0.4 cc aliquots may be injected along with a non-calcium product with control into animal model, for example, mice or rats. The implants will then be injected with either sodium thiosulfate or control in various concentrations and various injection timing protocols, beginning with weekly injections and titrating timing to effect. Evaluation of improvements in the removal of the exogenously placed calcium products to be determined both radiographically and histologically on volume. Optimal concentrations and local tissue safety will be evaluated.

### Example 2. Removal of Calcium Hydroxylapatite in a Porcine Experimental Model

Twelve porcine skin samples are obtained from the shoulder of a female white pig (aged approximately 1 year) within 15 minutes of sacrifice and preserved in formalin. Samples are injected subdermally with calcium hydroxylapatite (CaHA) using a 25-G needle with bolus volumes ranging from 0.4 to 0.8 mL. Injection sites are identified with a permanent marker pen. The samples are then randomized to 1 of 3 treatments: (1) topical sodium metabisulfite (SMB) (formulated in petrolatum jelly: 1-2 g and is applied topically, (2) 25% sodium metabisulfite in 120-mL gel) application with occlusion; (3) intralesional injection of 0.2-mL sodium thiosulfate (sodium thiosulfate) (concentration 12.5 g/50 mL), or both topical and intralesional treatments as described above. The sodium metabisulfite and sodium thiosulfate treatments are performed approximately 1 hour after calcium hydroxylapatite injection. In the control group, samples are not treated with sodium thiosulfate or sodium metabisulfite after CaHA injection. After approximately 24 hours, 4-mm punch biopsies of the treated areas may be taken for histologic processing and evaluation. Tissue samples can be fixed by direct immersion in a 10% formaldehyde solution before processing for light microscopy by dehydration, embedding in paraffin, and sectioning. The 5-mm thick sections are stained with hematoxylin and eosin to evaluate the presence, absence, or degradation of CaHA. Sections may be studied by light microscopy at magnifications of ×4, ×10, and ×40. A board-certified dermatopathologist who is blinded to control or treatment may review the specimens to estimate the amount of CaHA remaining in each sample.

### Example 3. Findings and Conclusions

This proof-of-concept study in a porcine model has been described and the results published in a recent journal article (Robinson, D. M. (2018) “In Vitro Analysis of the Degradation of Calcium Hydroxylapatite Dermal Filler: A Proof-of-Concept Study” Dermatol. Surg. 2018; (44):S5-S9, by the American Society for Dermatologic Surgery, Inc., the disclosure of this article is incorporated herein by reference for all purposes in its entirety.

The authors of the article demonstrated that intralesional sodium thiosulfate, injected either alone or in combination with topical sodium metabisulfite, is able to dissolve previously injected CaHA microspheres. The sodium thiosulfate was injected immediately after the injection of CaHA. When the authors evaluated the injection site of the CAHA/sodium thiosulfate, and on histologic analysis of the treated area 24 hours later, no visible CaHA microspheres were observed. Topical SMB was also found to be associated with CaHA degradation, but to a much lesser extent with approximately 50% of the CaHA remaining after topical treatment alone. The findings provided by this research article confirms the inventor's conception that intralesional sodium thiosulfate can be used to dissolve CaHA dermal filler when used for aesthetic purposes. Although correct injection technique, knowledge of anatomy, and filler selection mitigate the risk of many complications, errors can still occur, and unwanted CaHA and other calcium salts as described herein may require removal for medical or aesthetic reasons. For example, CaHA is indicated for subdermal implantation, and too superficial placement or injection into highly mobile areas may lead to visibility of the opaque material and nodularity. Other events such as vascular compromise are unpredictable and although rare can happen with any dermal filler even with the most experienced injector. The use of sodium thiosulfate and/or SMB can be used to reverse the effects of a calcium salt based filler (for example, CaHA) in the advent of an injection error or subsequent adverse effect. Although the above referenced journal article was conducted in a porcine model to demonstrate removal of dermal CaHA, previous research has demonstrated the feasibility of sodium thiosulfate use for dissolving other calcium salt deposits located in areas other than the skin or subdermal layers adjacent to skin in humans. (See for example, Gunasekera N S, et al. “Intralesional sodium thiosulfate treatment for calcinosis cutis in the setting of lupus panniculitis”. JAMA Dermatol 2017; 153:944-945; Del Barrio-Diaz P, et al. Topical sodium metabisulfite for the treatment of calcinosis cutis: a promising new therapy. Br J Dermatol 2016; 175:608-11; and Goossens J, et al. “Efficacy of intralesional sodium thiosulfate injections for disabling tumoral calcinosis: two cases.” Semin Arthritis Rheum 2017; 47:451-5.)

An ideal agent for dissolving a dermal filler would be well tolerated with minimal adverse effects (systemic or local). Sodium thiosulfate is on the World Health Association's model list of essential medicines as an intravenous agent for the treatment of cyanide poisoning, with which it binds to form a nontoxic thiocyanate, and as a topical agent (15% solution) for certain fungal infections. Sodium thiosulfate is classified by the FDA as “generally recognized as safe,” and there are no known contraindications. The exact mode of action of sodium thiosulfate has not been con-firmed, but is likely to be multifactorial. Proposed mechanisms include chelation of calcium into a calcium thiosulfate salt whose solubility is 250- to 100,000-fold higher than CaHA and induction of acidosis by modulation of local pH thereby increasing mineral solubility (the electrostatic bonds between calcium and phosphate ions are weakened by the reaction between hydrogen and phosphate ions). Dissolution of CaHA by either mechanism releases calcium and phosphate ions, which are safely removed through the body's normal physiological excretory processes.

The use of sodium thiosulfate for the removal of unwanted calcium salt such as calcium hydroxylapatite in the skin or subdermal layers has been effectively confirmed by the studies performed by others in the field. The published studies referenced herein confirms the feasibility of using intralesional sodium thiosulfate to dissolve CaHA in a porcine animal model in vitro.

While the present invention has been particularly shown and described with reference to the foregoing preferred and alternative embodiments, it should be understood by those skilled in the art that various alternatives to the embodiments of the invention described herein may be employed in practicing the invention without departing from the spirit and scope of the invention as defined in the following claims. It is intended that the following claims define the scope of the invention and that the method and apparatus within the scope of these claims and their equivalents be covered thereby. This description of the invention should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

1. A method for removing unwanted calcium salt deposited by injection cutaneously in a subject, the method comprises administering an effective amount of a composition containing sodium thiosulfate to the deposited calcium salt.

2. The method of claim 1, wherein the unwanted calcium salt is calcium hydroxylapatite.

3. The method of claim 2, wherein the unwanted calcium hydroxylapatite is RADIESSE®.

4. The method of claim 1, wherein the composition contains an amount of sodium thiosulfate ranging from about 0.001M to about 1.0 M.

5. The method of claim 4, wherein the composition contains an amount of sodium thiosulfate ranging from about 0.001M to about 0.5M sodium thiosulfate, and one or more cosmetic or pharmaceutically acceptable excipients.

6. The method of claim 5, wherein the sodium thiosulfate is formulated for topical application.

7. The method of claim 5, wherein the sodium thiosulfate is formulated for injection into one or more of the subject's epidermis, dermis, and hypodermis.

8. The method of claim 1, wherein the administration of sodium thiosulfate to the unwanted calcium salt provides a ratio of sodium thiosulfate to calcium salt (wt/wt) ranging from about 0.01 to 1 to about 1000 to 1.

9. The method of claim 1, wherein the sodium sulfate containing composition is administered after 1 to 30 minutes, after 1 hour, after 1 to 7 days, after 1-12 weeks, after 3-12 months or after 1 to 3 years, after the deposition of the calcium salt.

10. The method of claim 9, wherein the sodium thiosulfate containing composition is administered one to ten times topically, via injection or a combination of both at the site of the unwanted calcium salt.

11. The method of claim 1, wherein the unwanted calcium salt is deposited subdermally.

12. The method of claim 1, wherein the unwanted calcium salt is deposited in the epidermis, dermis, or hypodermis of the face, ears, or hands.

13. The method of claim 12, wherein the unwanted calcium salt is deposited in the face to correct wrinkles, folds, lips, or facial fat loss.

14. The method of claim 13, wherein a volume of about 0.01 mL to about 1.0 mL of unwanted calcium salt is injected intralesionally with about 0.1 mL to about 3.0 mL of the sodium thiosulfate containing composition, wherein the amount of sodium thiosulfate in the sodium thiosulfate containing composition ranges from about 1 g/50 mL to about 50 g/50 mL.

15. The method of claim 14, wherein the unwanted calcium salt to be removed is calcium hydroxylapatite.

16. The method of claim 15, wherein the calcium hydroxylapatite is administered in the form of RADIESSE®.
